(19) United States
(12) Patent Application Publication
(10) Pub. No.: US 2006/0068066 A1
(43) Pub. Date: Mar. 30, 2006

(54) Liquid composition and method for evaluating sorption behavior of flavor compounds using the liquid composition

(75) Inventor: Towa Kamishinbara, Minoh-shi (JP)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: Suntory Limited, Osaka-shi (JP)

(21) Appl. No.: 11/139,667

(22) Filed: May 31, 2005

(30) Foreign Application Priority Data
Sep. 24, 2004 (JP) 2004-277627

(51) Int. Cl. G01N 33/02

(52) U.S. Cl. 426/231

(57) ABSTRACT

A liquid composition according to the present invention contains, as sorption components, a hydrocarbon component, an ester component, an aldehyde component, an alcohol component, and a phenol component. These sorption components contain at least one compound having an evaporation energy-based solubility parameter of not less than 26.3. When this liquid composition is used to evaluate the sorption behavior of flavor compounds, it is possible to evaluate the sorption behavior of the flavor compounds, even in a packaging container using a high-polarity material such as PET, with a higher degree of reliability.

This Nonprovisional application claims priority under 35 U.S.C. § 119(a) on Patent Application No. 2004/277627 filed in Japan on Sep. 24, 2004, the entire contents of which are hereby incorporated by reference.

### FIELD OF THE INVENTION

The present invention relates to a liquid composition for use in quantifying an amount of its components sorbed by a given material, and use of the liquid composition. Particularly, the present invention relates to: the liquid composition which can be used suitably for the purpose of evaluating the sorption behavior of flavor compounds, and ensures a higher reliability of the evaluation; and a method for evaluating sorption behavior of flavor compounds using the liquid composition.

### BACKGROUND OF THE INVENTION

In general, sensory evaluation of foods are mainly based on taste. However, actually important are not only taste recognized when foods are put in a mouth but also a comprehensive sense obtained by complex interactions of various physical and chemical phenomena. Above all, aroma (flavor) is important in terms of taste.

Among foods, various drinks are in the form of liquid and therefore are more highly dependent in quality upon their flavors as well as tastes, as compared with solid foods. For drink manufacturers, it is ideal that flavors of drinks having designed compositions are kept unchanged until the drinks are put in consumers' mouths after undergoing a manufacturing operation and distribution. However, in many cases, flavors of the drinks change to some extent during processes undergone until the drinks come to hands of the consumers.

There are a wide variety of factors changing a flavor in a drink. Among these factors, one of the major factors is sorption of flavor compounds in a drink by a packaging container. Therefore, drink manufacturers must select packaging containers suitable for drinks for minimizing flavor changes. Among a wide variety of materials used for packaging containers, various synthetic resins have been recently used widely. On the sorption of flavor compounds by these synthetic resins, many investigations have been carried out, as indicated in non-patent documents 1 through 8 and patent document 1, for example. These documents disclose liquid compositions (sample solution, model flavor solution, or the like) for use in quantifying a sorption amount of flavor compounds.

More specifically, non-patent documents 1 and 2 disclose liquid compositions containing ester component, aldehyde component, and alcohol component. Non-patent documents 3 through 8 disclose liquid compositions containing hydrocarbon component, ester component, aldehyde component, and alcohol component. Patent document 1 discloses a liquid composition containing hydrocarbon component, ester component, alcohol component, and phenol component.

[Patent Document 1]

Japanese Patent No. 3504635 (Registered on Dec. 19, 2003; Japanese Laid-Open Patent Application No. 2002/361784; published on Dec. 18, 2002)

[Non-Patent Document 1]

M. Fukamachi, T. Matsui, Y. H. Hwang, M. Shimoda, and Y. Osajima, J. Agric. Food Chem., 44(9), 2810(1996)

[Non-Patent Document 2]

D. K. Arora, A. P. Hansen, and M. S. Armagost, J. Food Sci., 56(5), 1421 (1991)

[Non-Patent Document 3]

Tohru IKEGAMI, Mitsuya SHIMODA, and Yutaka OSAJIMA, Nippon Shokuhin Kogyo Gakkaishi Vol. 35, No. 7, p. 457 (1988)

[Non-Patent Document 4]

Tohru IKEGAMI, Mitsuya SHIMODA, and Yutaka OSAJIMA, Nippon Shokuhin Kogyo Gakkaishi Vol. 38, No. 5, p. 425 (1991)

[Non-Patent Document 5]

T. Ikegami, K. Nagashima, M. Shimoda, Y. Tanaka, and Y. Osajima, J. Food Sci., 56(2), 500(1991)

[Non-Patent Document 6]

Tohru IKEGAMI, Kazufumi NAGASHIMA, Mitsuya SHIMODA, and Yutaka OSAJIMA, Nippon Shokuhin Kogyo Gakkaishi Vol. 37, No. 10, p. 793 (1990)

[Non-Patent Document 7]

Tohru IKEGAMI, Mitsuya SHIMODA, Masayasu KOYAMA, and Yutaka OSAJIMA, Nippon Shokuhin Kogyo Gakkaishi Vol. 34, No. 5, p. 267 (1987)

[Non-Patent Document 8]

Ikuko NATSUHORI and Hiroaki SHIMADA, Nippon Shokuhin Kogyo Gakkaishi Vol. 41, No. 3, p. 173 (1994)

### SUMMARY OF THE INVENTION

However, the conventional techniques cannot sufficiently evaluate the sorption behaviors of flavor compounds by packaging containers using synthetic resin.

Specifically, the inventors of the present invention, from a study of various packaging containers using synthetic resin, have found that the liquid compositions disclosed in the conventional techniques contain various compounds as model flavor compounds respectively, but cannot sufficiently evaluate the sorption behaviors by packaging containers, according to the circumstances.

The present invention has been attained in view of the above problem, and an object of the present invention is to provide (I) a liquid composition which can be used suitably for the purpose of evaluating the sorption behavior of flavor compounds, and ensures a higher reliability of the evaluation, and (II) a method for evaluating sorption behavior of flavor compounds using the liquid composition.

The inventors of the present invention, as a result of extensive study in view of the above problem, have found the following fact on their own and brought the present invention to completion: for a highly reliable evaluation of the sorption of various flavor compounds by resin used for a drink container, it is effective to use a liquid composition containing, as essential components, the following five components: (i) hydrocarbon component, (ii) ester component, (iii) aldehyde component, (iv) alcohol component, and (v) phenol component.

That is, a liquid composition according to the present invention, in order to solve the above problem, is a liquid composition, which is brought into contact with a given material, for use in quantifying an amount of components sorbed by the material, wherein: an ester component, an aldehyde component, a hydrocarbon component, an alcohol component, and a phenol component are contained as sorption components sorption amounts of which are quantified.

The following description will sufficiently clarify further objects, characteristics, and excellent points of the present invention. Further, advantages of the invention will be clarified with reference to the ensuing detailed description taken in conjunction with the accompanying drawings.

### BRIEF DESCRIPTION OF THE DRAWINGS

FIG. 1A is a graph illustrating a relation between SP values and sorption amounts of sorption components (flavor compounds) in a PE film, according to Example 2.

FIG. 1B is a graph illustrating a relation between SP values and sorption amounts of sorption components (flavor compounds) in a PET film, according to Example 2.

FIG. 2 is a radar chart illustrating a relation between sorption components (flavor compounds) and sorption amounts in metal can's inner surface coating-use coatings A and B, according to Examples 3 and 4.

### DESCRIPTION OF THE EMBODIMENTS

The following will describe one embodiment of the present invention in detail. However, the present invention is not limited to the following description.

### Liquid Composition of the Present Invention

A liquid composition of the present invention contains water or lower alcohol aqueous solution as a solvent, and contains at least hydrocarbon component, ester component, aldehyde component, alcohol component, and phenol component, as solutes. These components should be compounds generally used as model flavor compounds. These components (solutes) are brought into contact with a given material for their sorption by the material, and sorption amount of the components are to be quantified. In the present invention, these components are therefore referred to as “sorption components” for convenience in description. In the present invention, it is preferable that as at least one compound of these solutes, a compound having an evaporation energy value-based solubility parameter of not less than 26.3 is used.

(1-1) Solvent

In the present invention, a solvent of the liquid composition, i.e. a solvent dissolving the foregoing sorption components should be, but not particularly limited to, a solvent that allows the foregoing sorption components to take the sorption behavior. Generally, at least any one of water, alcohol (lower alcohol) having not more than four carbon atoms, and saturated hydrocarbon having 5 to 17 carbon atoms can be preferably used. These solvents take a liquid form at room temperature (generally, in a temperature range from 15° C. to 25° C.) and are preferable because they are widely used as solvents in a variety of fields.

Among the foregoing solvents, water is more preferable to be of higher purity, and a distilled water, a ultrapure water, or the like is suitably used. Moreover, water can be used as a mixed solution with other solvent, i.e. an aqueous solution. Examples of the aqueous solution include, but not particularly limited to, a lower-alcohol aqueous solution.

The lower alcohols among the foregoing solvents are not particularly limited as far as they are alcohols having not more than four carbon atoms. More specifically, examples of the lower alcohols includes, but not particularly limited to, methanol, ethanol, 1-propanol(n-propanol), 2-propanol(isopropanol), 1-butanol (n-butanol), 2-butanol, tert-butyl alcohol, and others. These lower alcohols may be used singly or used in combination of two or more types thereof, or may be mixed with water to be used as an aqueous solution. Especially, in the present invention, ethanol is the most generally used among the foregoing lower alcohols since ethanol can be used preferably in the field of packaging for drinks.

If the lower alcohol is used as an aqueous solution, concentration of the lower alcohol, especially concentration of ethanol aqueous solution, is preferably in the range from 10 to 25 volume percent, more preferably approximately 20 volume percent. The ethanol aqueous solution in the above ranges can be used as a solvent that is sufficiently considered in view of influence on solubility of a solute (see Example 3 described later). Of course, if an aqueous solution contains alcohol other than ethanol, a concentration in this range is preferably adopted.

More specifically, examples of the saturated hydrocarbon having 5 to 17 carbon atoms include: linear alkane such as n-pentane, n-hexane, n-heptane, n-octane, n-nonane, n-decane, n-undecane, n-dodecane, n-tridecane, n-tetradecane, n-pentadecane, n-hexadecane, and n-heptadecane; branched chain alkane such as 2-methylpentane, 3-methylpentane, 2,2-dimethylbutane, and 2,3-dimethylbutane; and cyclic alkane such as cyclohexane and methylcyclohexane. These compounds may be used singly or used in combination of two or more types thereof. These compounds may be combined with other solvent capable of dissolving saturated hydrocarbons, such as a lower alcohol.

Needless to say, apart from the foregoing water, lower alcohol, or saturated hydrocarbon, a solvent to be used may be different depending upon a purpose for using the liquid composition.

(1-2) Model Compounds of Flavor Compounds as Sorption Components

In the present invention, as solutes, i.e. sorption components, of the liquid composition, various kinds of compounds used as flavor compounds can be used suitably. In the present invention, the following all five components are used as essential components: hydrocarbon component, ester component, aldehyde component, alcohol component (this is not the foregoing solvent.), and phenol component.

A liquid composition according to the present invention, by containing these five components, contains flavor compounds (sorption components) contained in various kinds of drinks such as whisky, tea, and fruit-flavor drink, or contains their relative compounds. This realizes highly reliable evaluation of the sorption behavior.

<Hydrocarbon Components>

Hydrocarbon components used in the present invention are not particularly limited as far as they can be used as flavors for various drinks and foods. More specifically, examples of the hydrocarbon component include: terpene such as α-pinene, limonene, and myrcene; and an aromatic compound such as cymene. These compounds may be used singly or used in combination of two or more types thereof as appropriate.

It is preferable that among these compounds, at least one of terpene and the aromatic compound are used. Above all, it is more preferable that α-pinene or limonene is contained, and it is particularly preferable that both α-pinene and limonene are contained. Many of these compounds are volatile substances contained in plants or the like and are important as flavor compounds. Therefore, these compounds can be suitably used as model flavor compounds. Note that, aldehyde component and alcohol component, which will be described later, also contain compounds that are contained in terpene. For example, neral, nerol, citral, citronellal, and menthol are categorized as aldehyde components or alcohol components. However, they are terpenes. However, the present embodiment assumes, for convenience in description, that compounds having (alcohol) hydroxyl group and aldehyde group are terpenes, but not included in the category of hydrocarbon component.

<Ester Components>

The ester components used in the present invention are not particularly limited as far as they are compounds that have an ester linkage in its structure and can be used as flavors for various drinks and foods. More specifically, examples of the ester components include: acetic acid ester such as ethyl acetate, butyl acetate, amyl acetate, and isoamyl acetate; butyric acid ester such as ethyl butyrate, propyl butyrate, butyl butyrate, amyl butyrate, and isoamyl butyrate; lauric acid (dodecanoic acid) ester such as ethyl laurate; myristic acid (tetradecanoic acid) ester such as ethyl myristate; valeric acid (pentanoic acid) ester such as ethyl valerate; caproic acid (hexanoic acid) ester such as methyl caproate and ethyl caproate; oenanthic acid (heptanoic acid) ester such as ethyl oenanthate; caprylic acid (octanoic acid) ester such as ethyl caprylate; pelargonic acid (nonanoic acid) ester such as ethyl pelargonate; capric acid (decanoic acid) ester such as ethyl caprate; 2-methyl butyric acid ester such as ethyl 2-methyl butyrate; isovaleric acid ester such as ethyl isovalerate and isoamyl isovalerate; benzoic acid ester such as methyl benzoate, ethyl benzoate, propyl benzoate, butyl benzoate, and amyl benzoate; lactic acid ester such as ethyl lactate, cis-3-hexenyl lactate, trans-3-hexenyl lactate, and benzyl lactate; propionic ester such as methyl propionate, ethyl propionate, butyl propionate, allyl propionate, and cyclohexyl propionate; and cinnamic acid ester such as methyl cinnamate and ethyl cinnamate; anthranilic acid ester such as methyl anthranilate. These compounds may be used singly or used in combination of two or more types thereof as appropriate.

It is preferable that among the foregoing compounds, at least one of the following compounds: ethyl acetate, ethyl butyrate, ethyl caproate, ethyl caprate, ethyl laurate, ethyl myristate, and methyl anthranilate are contained in the ester components. It is particularly preferable that ethyl acetate, ethyl butyrate, ethyl caproate, ethyl caprate, ethyl laurate, ethyl myristate, and methyl anthranilate are all contained in the ester components.

<Aldehyde Components>

Aldehyde components used in the present invention are not particularly limited as far as they are compounds that have an aldehyde group in their structure and can be used as flavors for various drinks and foods. More specifically, examples of the aldehyde components include: saturated hydrocarbon-based aldehydes such as butanal, pentanal, hexanal, trans-2-hexanal, 2-ethylhexanal, heptanal, nonanal, octanal, decanal, undecanal, dodecanal, and isobutyraldehyde; unsaturated hydrocarbon-based aldehydes such as geranial, citral (cis and trans), citronellal, neral, and perilla aldehyde; aromatic aldehydes such as benzoaldehyde, cinnamaldehyde, anisaldehyde, and phenylpropionic aldehyde. These compounds may be used singly or used in combination of two or more types thereof as appropriate.

It is preferable that among the foregoing compounds, at least one of the following compounds: citral, iso-butanal, n-butanal, n-octanal, n-dodecanal, and benzaldehyde are contained in the foregoing aldehyde components. It is particularly preferable that citral, iso-butanal, n-butanal, n-octanal, n-dodecanal, and benzaldehyde are all contained in the foregoing aldehyde components.

<Alcohol Components>

Alcohol components used as sorption components in the present invention are not particularly limited as far as they are compounds that have a hydroxyl group in their structure and can be used as flavors for various drinks and foods. Note that, there can exist overlapping compounds between the alcohol components listed below and the lower alcohols suitably used as the solvents. However, they play different roles and will be therefore regarded as different components.

More specifically, examples of the foregoing alcohol components include: saturated hydrocarbon-based alcohol such as n-butanol, iso-butanol, hexanol, isoamyl alcohol, n-heptanol, n-heptanol, n-octanol, n-nonanol, n-decanol, n-undecanol, n-dodecanol, and menthol; unsaturated hydrocarbon-based alcohols such as cis-3-hexen-1-ol, geraniol, linalool, citrol, nerol, and terpineol; and aromatic alcohols such as benzyl alcohol and methyl eugenol. These compounds may be used singly or used in combination of two or more types thereof as appropriate.

It is preferable that among the above compounds, at least one of the following compounds: linalol, geraniol, iso-butanol, n-butanol, n-octanol, n-dodecanol, and benzyl alcohol are contained in the foregoing alcohol components. It is particularly preferable that linalol, geraniol, iso-butanol, n-butanol, n-octanol, n-dodecanol, and benzyl alcohol are all contained in the foregoing alcohol components.

<Phenol Components>

The phenol components used in the present invention are not particularly limited as far as they are compounds that have a phenolic hydroxy group in their structure and can be used as flavors for various drinks and foods. In the broad sense, it can be assumed that phenol components are included in the foregoing alcohol components. However, the phenol components and the alcohol components are different in chemical properties, and play different roles as the sorption components (flavor compounds) in the present invention. Therefore, they are regarded as ones that are different from the alcohol components.

More specifically, examples of the foregoing phenol components include: phenol, cresol, hydroquinone, and catechol. These compounds may be used singly or used in combination of two or more types thereof as appropriate. Among these compounds, phenol or cresol is preferable, and phenol is more preferable. It is particularly preferable that both phenol and cresol are contained. Phenol and cresol each has a high polarity, as will be described later, and can be suitably used as a compound having an evaporation energy value-based solubility parameter of not less than 26.3 in the present invention.

<Other Sorption Components>

A liquid composition according to the present invention may contain sorption components other than the foregoing five types of components. The other sorption components are not particularly limited as far as they are compounds that can be used as flavors for various drinks and foods. More specifically, examples of other sorption components include: ketones such as 2-heptanone, 2-octanone, 2-nonanone, and 2-decanone; sulfur compounds such as 2-methylthiopropanol, 3-methylthiophen, and benzyl methyl sulfide; and nitrogen compounds such as methylpyrazine and ethylpyrazine. These compounds may be used singly or used in combination of two or more types thereof as appropriate.

<Concentration of Sorption Components>

As to a liquid composition according to the present invention, a final concentration of each of the compounds used as the sorption components (flavor compounds), i.e. the hydrocarbon component, ester component, aldehyde component, alcohol component, phenol component, and other sorption components is not particularly limited, but is preferably in the range from 1 ppm to 100 ppm, more preferably approximately 10 ppm. Concentration of each compound each in this concentration range means substantially the same concentration of a flavor compound contained in a general drink. Therefore, a liquid composition according to the present invention can be used suitably as a model solution for a drink.

(1-3) Components Other Than Sorption Components

A liquid composition according to the present invention may contain, as a solute, a component other than the foregoing sorption components. Such other solute is not particularly limited. Specifically, examples of the other solute include a solubilizing agent of the sorption components (flavor compounds). More specifically, as such a solubilizing agent, polyalcohol-type nonionic surfactant can be used suitably.

In many cases, as described earlier, the sorption components are of low hydrophilicity and therefore resist dissolving in water or alcohol aqueous solution. However, the use of the polyalcohol-type nonionic surfactant allows the sorption components to be sufficiently dissolved therein. Accordingly, it is possible to obtain a model solution closer to a drink. The polyalcohol-type nonionic surfactant is often used as a surfactant for foods and therefore is suitable as a component added to a model solution for a drink.

More specifically, examples of the polyalcohol-type nonionic surfactant include sugar ester, glycerin fatty acid ester, sorbitan fatty acid ester, and propyleneglycol fatty acid ester. These compounds may be used singly or used in combination of two or more types thereof as appropriate. Note that, sugar ester is used in Examples described later.

The amount of the solubilizing agent to be added, which is not particularly limited, should be the amount that allows the sorption components to be sufficiently dissolved in the solvent. More specifically, the amount of the solubilizing agent should be in the range from 0.01% to 1% (w/v). The amount of the solubilizing agent in this range allows the sorption components to be sufficiently dissolved in the solvent and avoids the occurrence of the event that sorption components take out-of-ordinary sorption behavior, as compared with a model for a drink, due to too much amount of the solubilizing agent.

(1-4) Manufacturing Method of Liquid Composition

A manufacturing method of the liquid composition according to the present invention is not particularly limited, but should be prepared by mixing the foregoing components. The order of the mixture is not particularly limited. However, since absolute amount of the sorption components is small, the mixture should be performed in such an order that the solvent is first prepared, and thereafter the sorption components are added to the solvent. The preparation of the solvent herein means preparation of a desired amount of water, if the solvent is water only. If the solvent is alcohol aqueous solution, alcohol and water in predetermined amounts should be mixed so that a desired concentration can be obtained.

If it is necessary to add the solubilizing agent, the solubilizing agent in a predetermined amount should be added and dissolved in the solvent in advance. If necessary, a mixture solution of the solubilizing agent and the solvent may be heated so that the solubilizing agent can be dissolved sufficiently. Heating temperature is not particularly limited, but should be in such a temperature range where water and/or alcohol do not evaporate excessively.

(2) Solubility Parameters of Sorption Components Contained in the Liquid Composition

In the present invention, solutes used as the sorption components, as described above, preferably contain at least one compound having an evaporation energy-based solubility parameter of not less than 26.3 Mpa^{1/2}. The evaporation energy-based solubility parameter (solubility parameter: SP value) herein is calculated from an evaporation energy value disclosed in R. F. Fedors, Polym. Eng. Sci., 14, 147 (1974).

More specifically, assuming that an evaporation energy is E_{coh} (J/mol), a molecular volume is V (cm³/mol), the sum of the evaporation energies E_{coh} of all groups contained in the compound whose SP value is to be calculated is ΣE_{coh}, and the sum of the molecular volumes V of all groups contained in a compound whose SP value are to be calculated is ΣV, a SP value δv is calcuated by the following equation (1):

$${δ}_{v} = {( \frac{\sum {E}_{coh}}{\sum V} )}^{\frac{1}{2}} \qquad ( 1 )$$

Note that, for specific values of the evaporation energy E_{coh} and molecular volume V of the groups contained in the compound whose SP value is to be calculated, values described in a publicly known document (e.g. Van Krevelen, D. W. “Cohesive Properties and Solubility”; In Properties of Polymers, Elsevier Scientific Publishing, Netherlands, Chapter 7 (1997)) can be used.

Various drinks are filled in packaging containers made of various materials. When a packaging container using synthetic resin and filled with a drink is regarded as one system, the packaging container is made up of flavor compound, synthetic resin (packaging container), and solvent (drink). In the sorption phenomenon in this packaging container, affinity between a flavor compound and synthetic resin becomes an important factor. In view of this, in the present invention, a high-polarity compound having a SP value of not less than 26.3 Mpa^{1/2} is used as a sorption component (flavor compound). This allows the sorption behavior of a flavor compound to be accurately evaluated regardless of what substance and material are used for the solvent and the packaging container. As a result, the sorption behavior of a flavor compound can be evaluated with a higher degree of reliability.

(2-1) SP Value of High-Polarity Compound Contained in Liquid Composition

As described above, a liquid composition according to the present invention should contain at least one compound having a SP value of not less than 26.3 MPa^{1/2}. Such a compound may be contained in any one of the foregoing essential five components, but should be a compound contained in phenol components, for example. More specifically, in the above example, examples of such a compound include cresol (SP value: 26.3 MPa^{1/2}) and phenol (SP value: 27.5 Mpa^{1/2}). More preferably, phenol is more preferably used, particularly judging from its high SP value.

Further, when the foregoing compound having an SP value of not less than 26.3 MPa^{1/2} is defined as “upper-level high-polarity compound”, a liquid composition according to the present invention preferably contains at least one “middle-level high-polarity compound” as well as the foregoing “upper-level high-polarity compound”. The “middle-level high-polarity compound” herein is defined as a compound having an SP value of not less than 22.0 MPa^{1/2}.

Such a “middle-level high-polarity compound” is also used in the conventional liquid compositions (For example, Non-Patent documents 6 and 7). Therefore, the use of the “middle-level high-polarity compound” together with the “upper-level high-polarity compound” ensures evaluation of the sorption behavior with a higher degree of reliability.

The “middle-level high-polarity compound” should be contained in any one of the foregoing essential five components. For example, it should be contained in at least any one of ester component, aldehyde component, and alcohol component. More specifically, examples of the ester component, aldehyde component, and alcohol component include iso-butanol (alcohol component), n-butanol (alcohol component), methyl anthranilate (ester component), benzaldehyde (aldehyde component), and benzyl alcohol (alcohol component).

For example, in the technique disclosed in Patent Document 1, phenol, an example of the “upper-level high-polarity compound”, is used in a model solution. However, in this technique, the model solution does not contain an aldehyde component and is therefore used as an effective model solution in a technical scope disclosed in the Patent Document 1, but is insufficient for the use in comprehensively evaluating the sorption behavior of the foregoing flavor compounds.

On the contrary, a liquid composition according to the present invention uses, as the sorption components (flavor compounds), the following five components: hydrocarbon component; ester component; aldehyde component; alcohol component; and phenol component. This combination covers compounds that belong to typical categories of flavor compounds contained in drinks, and contains the foregoing “upper-level high-polarity compound”. Therefore, the present invention allows the sorption behavior of flavor compounds to be evaluated more comprehensively and with a higher degree of reliability.

(2-2) SP Value of Each Component

In the present invention, as the sorption components (flavor compounds), contained are the following five components: hydrocarbon component, ester component, aldehyde component, alcohol component, and phenol component. Among these components, the phenol component, which is suitably used as “upper-level high-polarity compound”, should have a SP value of not less than 26.3 MPa^{1/2}, including 27.5 MPa^{1/2} (phenol). Also, there are preferable SP value ranges for the other components.

Specifically, a compound used as the hydrocarbon component, i.e. a compound contained in the hydrocarbon component of the liquid composition preferably has the SP value in the range from 16.2 MPa^{1/2} to 18.5 MPa^{1/2}. Further, a compound used as the ester component, i.e. a compound contained in the ester component of the liquid composition preferably has the SP value in the range from 18.0 MPa^{1/2} to 23.6 MPa^{1/2}. Still further, a compound used as the aldehyde component, i.e. a compound contained in the aldehyde component of the liquid composition preferably has the SP value in the range from 18.7 MPa^{1/2} to 23.8 MPa^{1/2}. Yet further, a compound used as the alcohol component, i.e. a compound contained in the alcohol component of the liquid composition preferably has the SP value in the range from 20.1 MPa^{1/2} to 26.2 MPa^{1/2}.

That the SP values of the above components fall within the foregoing ranges means that compounds that belong to typical categories of flavor compounds contained in drinks can be covered, and that polarities of compounds in the respective categories fall within the ranges of the polarities of general flavor compounds. Therefore, the present invention allows the sorption behavior of flavor compounds to be evaluated more comprehensively and with a higher degree of reliability.

From a broad view, a liquid composition according to the present invention contains, as the sorption components, hydrocarbon component, ester component, aldehyde component, alcohol component, and phenol component, and the SP value of each sorption component should fall within the range from 17.6 MPa^{1/2} to 27.5 MPa^{1/2}. Note that, in order to evaluate the sorption behavior more accurately, it is very preferable that a liquid composition according to the present invention contains the “upper-level high-polarity compound” having the SP value of not less than 26.3 MPa^{1/2}.

(3) Use of the Present Invention

Specifically, a liquid composition according to the present invention can be adopted more suitably for use in evaluating the sorption behavior of drink's flavor compounds in a packaging container using synthetic resin. Of course, the present invention is not limited to this use. The use of a liquid composition according to the present invention is not limited to the use in the fields of packaging containers and drinks as far as the liquid composition is brought into contact with a given material, for use in quantifying the amount of components sorbed by the material.

(3-1) Components' Sorption Quantified Material

A components'-sorption-quantified material (for convenience of explanation, hereinafter referred to as “sorption-quantified material”) is not particularly limited, but examples of the sorption-quantified material in the present invention include general synthetic resins for a wide variety of uses as packaging containers for drinks. More specifically, examples of the sorption-quantified material include: polyester resin such as polyethylene terephthalate (PET); polyolefine resin such as polyethylene (PE); vinyl resin such as ethylene-vinyl alcohol copolymer (EVOH); and polyamide (PA).

Particularly, a liquid composition according to the present invention contains the foregoing five components and contains the upper-level high-polarity compound. Therefore, even if the sorption-quantified material is a material having a high polarity (high-polarity material), such as PET having an ester linkage in its structure and PA having acid-amide linkage in its structure, it is possible to evaluate the sorption behavior of flavor compounds more comprehensively and with a higher degree of reliability.

A specific structure of the sorption-quantified material of the present invention is not particularly limited, but the sorption-quantified material of the present invention is preferably in plate form or film form so as to be used suitably for a packaging container. Further, this sorption-quantified material in plate form or film form is not necessarily in the form of plate or film made of synthetic resin only as far as it has a structure containing synthetic resin. For example, the sorption-quantified material may be of a laminated structure in which non-synthetic resin material such as paper or metal is laminated on synthetic resin. In alternative example, the sorption-quantified material may be of a laminated structure of synthetic resins. In another alternative example, the sorption-quantified material may be of a polymer-blended structure having plural types of synthetic resins mixed.

(3-2) Specific Technique Using the Present Invention

A more specific technique using the present invention is not particularly limited, but examples of the technique using the present invention include a method for evaluating the sorption behavior of flavor compounds, including: a contact step of bringing the liquid composition into contact with a given material; and a sorption-amount measuring step of measuring an amount of liquid composition's solute sorbed by the material.

The contact step is not particularly limited as far as it allows a liquid composition according to the present invention to be brought into contact with a given material so that flavor compounds (sorption components) can be sorbed by the material. In Examples described later, a film cut into a desired size is hermetically immersed in the liquid composition and is stored until sorption reaches saturation. However, the present invention is not limited to this. Alternatively, depending upon use conditions of a the material, the film may be released from contact with the liquid composition before sorption reaches saturation, or may immersed in the liquid composition while being exposed to air.

The sorption-amount measuring step is not particularly limited as far as it can measure (quantify) the sorption amount of components by the material released from contact. In Examples described later, sorbed components are extracted with diethyl ether, concentrated, and then analyzed by gas chromatography (GC). However, the present invention is not limited to this.

The shape of a material (sample) used for evaluation of sorption behavior of the sorption components is not particularly limited, but a film-shaped product can be generally taken as an example. This is the shape suitable for packaging containers such as a PET bottle. Of course, the present invention is not limited to this. Alternatively, depending upon use conditions of the material, a block-shaped product may be used as the sample.

Further, the present invention can include a kit for carrying out the foregoing sorption behavior evaluation method. Specific components that make up the kit are not particularly limited, but the kit should include at least liquid composition. In addition, the kit may include a film as a sample that controls sorption behavior and a solvent (aforementioned diethyl ether, or others) with which components are extracted from the sample.

As described above, a liquid composition according to the present invention, in order to solve the above problem, is a liquid composition, which is brought into contact with a given material, for use in quantifying an amount of components sorbed by the material, wherein: a hydrocarbon component, an ester component, an aldehyde component, an alcohol component, and a phenol component are contained as sorption components sorption amounts of which are quantified.

In the foregoing liquid composition, it is preferable that the hydrocarbon component contains at least one of terpene and an aromatic compound. It is more preferable that the hydrocarbon component contains α-pinene or limonene. It is particularly preferable that the hydrocarbon component contains both α-pinene and limonene. Further, it is preferable that the ester component contains at least one of the following compounds: ethyl acetate, ethyl butyrate, ethyl caproate, ethyl caprate, ethyl laurate, ethyl myristate, and methyl anthranilate. It is particularly preferable that the ester component contains all of the following compounds: ethyl acetate, ethyl butyrate, ethyl caproate, ethyl caprate, ethyl laurate, ethyl myristate, and methyl anthranilate. Further, it is preferable that the aldehyde component contains at least one of the following compounds: citral, iso-butanal, n-butanal, n-octanal, n-dodecanal, and benzaldehyde. It is particularly preferable that the aldehyde component contains all of the following compounds: citral, iso-butanal, n-butanal, n-octanal, n-dodecanal, and benzaldehyde. Still further, it is preferable that the alcohol component contains at least one of the following compounds: linalol, geraniol, iso-butanol, n-butanol, n-octanol, n-dodecanol, and benzyl alcohol. It is particularly preferable that the alcohol component contains all of the following compounds: linalol, geraniol, iso-butanol, n-butanol, n-octanol, n-dodecanol, and benzyl alcohol. Yet further, it is preferable that the phenol component contains phenol or cresol. It is particularly preferable that the phenol component contains both phenol and cresol.

In the foregoing liquid composition, it is preferable that at least one compound having an evaporation energy-based solubility parameter of not less than 26.3 MPa^{1/2} is further contained. In addition, it is particularly preferable that at least one compound having the evaporation energy-based solubility parameter of not less than 22.0 MPa^{1/2} is contained. The compound having the solubility parameter of not less than 26.3 MPa^{1/2} should be contained in the phenol component, and the compound having the solubility parameter of not less than 22.0 MPa^{1/2} should be contained in at least any one of the ester component, the aldehyde component, and the alcohol component. However, the present invention is not particularly limited to these arrangements.

In the foregoing liquid composition, a compound contained in the alcohol component preferably has the solubility parameter in a range from 20.1 MPa^{1/2} to 26.2 MPa^{1/2}, a compound contained in the ester component preferably has the solubility parameter in a range from 18.0 MPa^{1/2} to 23.6 MPa^{1/2}, a compound contained in the aldehyde component preferably has the solubility parameter in a range from 18.7 MPa^{1/2} to 23.8 MPa^{1/2}, and a compound contained in the hydrocarbon component preferably has the solubility parameter in a range from 16.2 MPa^{1/2} to 18.5 MPa^{1/2}.

Further, in the foregoing liquid composition, it is preferable that as a solvent dissolving the sorption components, at least any one of water, alcohol having not more than four carbon atoms, and saturated hydrocarbon having 5 to 17 carbon atoms is used.

Still further, in the foregoing liquid composition, it is preferable that as a solubilizing agent for the sorption components, a polyalcohol-type nonionic surfactant is further contained. As the polyalcohol-type nonionic surfactant, at least any one of sugar ester, glycerin fatty acid ester, sorbitan fatty acid ester, and propyleneglycol fatty acid ester can be used suitably.

Yet further, in the foregoing liquid composition, it is preferable that compounds used as the sorption components are dissolved in a solvent so as to make a final concentration of each of the compounds in a range from 1 ppm to 100 ppm.

A material in which sorption amount of components is to be quantified using the foregoing liquid composition is not particularly limited, but synthetic resin can be taken as an example of the material. More specifically, examples of the synthetic resin include any one of polyethylene terephthalate (PET), polyethylene (PE), ethylene-vinylalcohol copolymer (EVOH), and polyamide (PA).

Further, in the present invention, a method for evaluating sorption behavior of flavor compounds, comprising: a contact step of bringing the foregoing liquid composition into contact with a given material; and a sorption-amount measuring step of measuring an amount of a solute of the liquid composition sorbed by the material can be taken. A given material used in this method is preferably in film form. The foregoing method may come in kit form.

In the present invention, as described above, the liquid composition contains (i) a hydrocarbon component, (ii) an ester component, (iii) an aldehyde component, (iv) an alcohol component, and (v) a phenol component.

Conventionally, it is known that a combination of two to four components among the foregoing components (i)-(v) is used for a liquid composition. However, the liquid composition containing all of the foregoing five components is not known. Specifically, none of the liquid compositions disclosed in non-patent documents 1 and 2 contain (i) hydrocarbon component and (v) phenol component. None of the liquid compositions disclosed in non-patent documents 3 through 8 contain (v) phenol composition. The liquid composition disclosed in patent document 1 does not contain (iii) aldehyde component. This is because no consideration is conventionally given to a balance of the combination of plural flavor compounds that ensures an excellent evaluation in terms of affinity between flavor compounds and synthetic resin.

On the contrary, a liquid composition of the present invention contains the foregoing five components that are essential as model flavor compounds. Further, the liquid composition is used, containing combined these five components respectively having solubility parameters ranges of which are specified as needed. This achieves a greater general versatility for various model drinks and ensures a comprehensive evaluation of the sorption behavior of flavor compounds. As a result, for example, this brings the effect of evaluating the sorption behavior of flavor compounds with a higher degree of reliability even in a variety of packaging containers.

### EXAMPLES

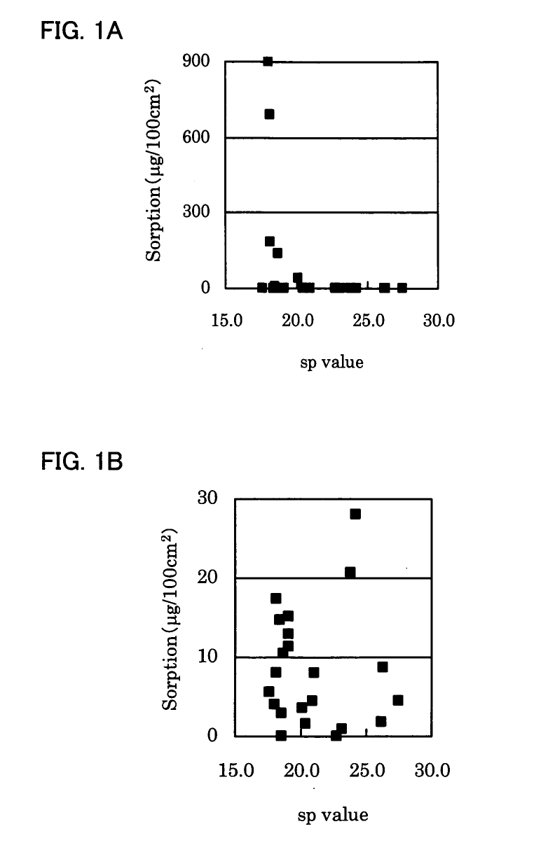

The following will more specifically describe the present invention with reference to Examples and FIG. 1. However, the present invention is not limited to this description. The present invention is susceptible of various changes, variations, and modifications by a person skilled in the art within the scope of the present invention.

[Reagents]

As a distilled water, high performance liquid chromatography-use distilled water (manufactured by Wako Pure Chemical Industries, Ltd.) was used. As ethanol, precise analysis-use ethanol (manufactured by Wako Pure Chemical Industries, Ltd.) was used. As sorption components (flavor compounds), special grade regents (manufactured by Wako Pure Chemical Industries, Ltd. and Kanto Chemical Co., Inc.) were used. In collecting sorption components (flavor compounds), residual pesticide analysis-use diethyl ether (manufactured by Wako Pure Chemical Industries, Ltd.) was used.

[Collection of Sorption Components Sorbed by Film]

In Examples given below, sorption components were collected from a film in the following manner: A film immersed in a liquid composition (model solution) was taken out of the liquid composition and washed twice with 100 ml of distilled water, and thereafter, water on the surface of the film was dried in air. The film immersed in hexane solution was volatilized hexane on the surface of the film. The film subjected to sorption in vapor phase was used as it is. The obtained film was cut into small pieces, put in an Erlenmeyer flask having 50 ml capacity, and immersed in 50 ml of residual pesticide analysis-use diethyl ether. The small pieces of film were immersed for three days while a solvent in the flask was repeatedly replaced, and sorbed components were extracted. A total extract was collected and concentrated by KD concentration technique to make 1 ml to 5 ml of extract.

The concentrated extract was analyzed by gas chromatography (GC). GC conditions are as follows:

Instrument: Agilent HP6890 (product name, manufactured by Yokogawa Analytical Systems, Inc.)

Detector: FID

Column: HP-INNOWax (50 m×0.32 mm, 0.5 μm)

Oven temperature: 40° C. (5 min)−10° C./min−100° C.−5° C./min−220° C. (5 min)

Carrier gas: Herium

Inlet pressure: 76 kPa

Injection: 1 μl, split (10:1)

Inlet temperature: 220° C.

Detection temperature: 240° C.

### Example 1

To a distilled water or ethanol aqueous solution, 0.1% (w/v) of Ryoto Sugar Ester P-1570 (HLB value: 15) (product name, manufactured by Mitsubishi Kagaku Foods Corporation) was added as a solubilizing agent, and a resultant solution was stirred for dissolution while being heated. To the obtained aqueous solution, ethanol solution containing sorption components was added to make a 10 ppm final concentration of each component. This produces a liquid composition (model solution) according to the present invention. Added components (sorption components) were 22 substances of respectively different polarities, selected from among components contained in drinks. Respective solubility parameters (SP values) of the selected sorption components were calculated from the evaporation energy value of Fedors (see Section (2)). A list of the sorption components is shown in Table 1.

TABLE 1

| No. | Compounds | sp values | Components |
|---|---|---|---|
| 1 | Limonene | 17.6 | hydrocarbon |
| 2 | ethyl tetradecanoate | 18.0 | ester |
| 3 | ethyl dodecanoate | 18.1 | ester |
| 4 | ethyl decanoate | 18.1 | ester |
| 5 | ethyl hexanoate | 18.4 | ester |
| 6 | α-pinene | 18.5 | hydrocarbon |
| 7 | ethyl butyrate | 18.6 | ester |
| 8 | n-dodecanal | 18.7 | aldehyde |
| 9 | citral (cis + trans) | 19.1 | aldehyde |
| 10 | n-octanal | 19.1 | aldehyde |
| 11 | n-dodecanol | 20.1 | alcohol |
| 12 | n-butanal | 20.2 | aldehyde |
| 13 | Linalool | 20.4 | alcohol |
| 14 | Geraniol | 20.9 | alcohol |
| 15 | n-octanol | 21.0 | alcohol |
| 16 | i-butanol | 22.7 | alcohol |
| 17 | n-butanol | 23.2 | alcohol |
| 18 | methyl anthranilate | 23.6 | ester |
| 19 | Benzaldehyde | 23.8 | aldehyde |
| 20 | benzyl alcohol | 26.2 | alcohol |
| 21 | o-cresol | 26.3 | phenol |
| 22 | Phenol | 27.5 | phenol |

### Example 2

300 ml of the liquid composition (model solution) of Example 1 was poured into a hard-glass separable flask. A LDPE film cut into a 100 cm² (5 cm×20 cm) size (density: 0.920 g/cm², film thickness: 50 μm) and a PET film (crystallinity: 32%, film thickness: 50 μm) were immersed in the liquid composition, and the flask was covered with a lid and sealed with a fluorocarbon resin tape and an aluminum tape. Then, the PE film was stored at 35° C. for 25 days, and the PET film was stored at 35° C. for 35 days until sorption reaches saturation. Thereafter, the films were taken out for collection of sorption components, as described earlier. A plotting result indicating a relation between SP values and sorption amounts of the sorption components is shown in FIGS. 1A and 1B.

As shown in FIGS. 1A and 1B, a liquid composition according to the present invention completely contains components that are likely to be sorbed by both PE and PET. Therefore, it became clear that a liquid composition containing hydrocarbon component, ester component, aldehyde component, alcohol component, and phenol component is preferable for use in evaluating the sorption behavior regardless of whether PE or PET is used.

Thus, by using a liquid composition containing, as essential components, the five components: hydrocarbon component, ester component, aldehyde component, alcohol component, and phenol component, it is possible to more accurately evaluate the sorption behavior of flavor compounds. As a result, the sorption behavior of flavor compounds can be evaluated with a higher degree of reliability regardless of what substance and material are used for the solvent and the packaging container.

### Example 3

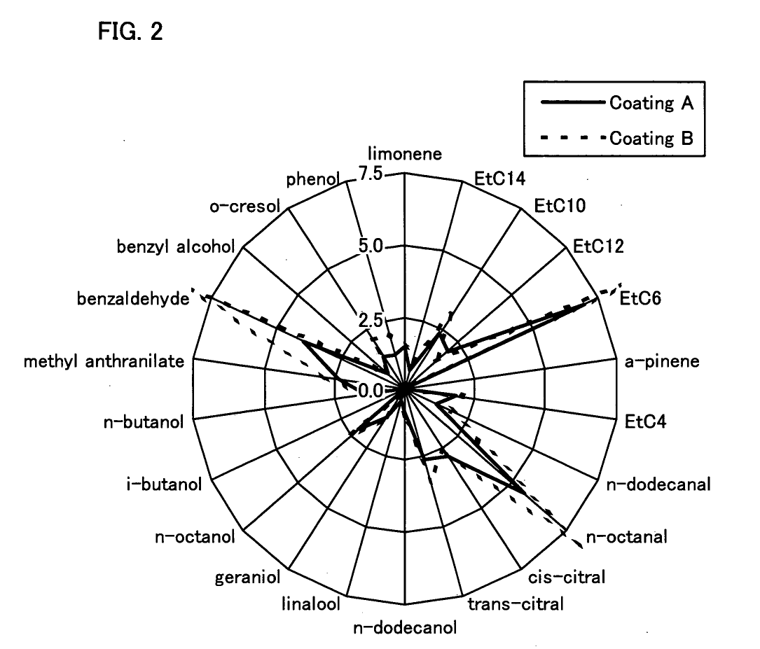

A liquid composition (model solution) was prepared in the same manner as Example 1, except for exclusion of n-butanal. A coating plate having a metal can's inner surface coating-use coating A (manufactured by Valspar Rock Co., Ltd.) made of a copolymer of epoxy resin, vinyl chloride resin, vinyl acetate resin, and phenol resin coated thereon was immersed in the foregoing liquid composition and stored at 35° C. for one month in the same manner as in Example 2. Thereafter, the film was taken out, and sorption components were collected from the film, as described earlier. Then, differences in sorption behavior by the coating A were evaluated. Sorption amounts of the components are shown in Table 2 and FIG. 2.

### Example 4

Differences in sorption behavior by the coating B were evaluated in the same manner as Example 3, except for the use of a metal can's inner surface coating-use coating B (manufactured by Valspar Rock Co., Ltd.) made of a copolymer of epoxy resin, acrylic resin and phenol resin. Sorption amounts of the components are shown in Table 2 and FIG. 2.

TABLE 2

| | | | Sorption by | Sorption by |
|---|---|---|---|---|
| | | | coating A | Coating B |
| No. | Compounds | Components | (μg/100 cm²) | (μg/100 cm²) |
| 1 | Limonene | hydrocarbon | 1.5 | 1.5 |
| 2 | ethyl tetradecanoate | ester | 0.7 | 1.1 |
| 3 | ethyl dodecanoate | ester | 2.3 | 3.0 |
| 4 | ethyl decanoate | ester | 2.0 | 1.5 |
| 5 | ethyl hexanoate | ester | 7.0 | 9.6 |
| 6 | α-pinene | hydrocarbon | 0.0 | 0.0 |
| 7 | ethyl butyrate | ester | 1.8 | 2.0 |
| 8 | n-dodecanal | aldehyde | 1.2 | 1.8 |
| 9 | citral (cis + trans) | aldehyde | 5.5 | 8.3 |
| 10 | n-octanal | aldehyde | 2.8 | 2.5 |
| 11 | n-dodecanol | alcohol | 2.6 | 3.4 |
| 12 | linalool | alcohol | 0.8 | 1.0 |
| 13 | geraniol | alcohol | 0.4 | 0.4 |
| 14 | n-octanol | alcohol | 1.2 | 1.3 |
| 15 | i-butanol | alcohol | 2.2 | 2.4 |
| 16 | n-butanol | alcohol | 0.0 | 0.0 |
| 17 | methyl anthranilate | ester | 1.4 | 1.4 |
| 18 | benzaldehyde | aldehyde | 2.3 | 2.9 |
| 19 | benzyl alcohol | alcohol | 3.9 | 8.3 |
| 20 | o-cresol | phenol | 0.8 | 0.9 |
| 21 | Phenol | phenol | 1.4 | 2.0 |

As is apparent from the results of Examples 3 and 4, a liquid composition according to the present invention completely contains components that are likely to be sorbed by the metal can's inner surface coating-use coatings, too. Therefore, it became clear that a liquid composition according to the present invention is preferable for use in evaluating the sorption behavior by coatings.

As described above, a liquid composition according to the present invention ensures evaluation of the sorption behavior of sorption components, with a higher degree of reliability, even in a packaging container using a high-polarity material, for example. When consideration is given to a drink contained in a PET bottle, for example, there are differences in behavior between sorption of foreign odor or the like in a vapor phase outside the bottle and sorption of components in a drink contained in the bottle. Therefore, the present invention can have a wide variety of applications such as a future development of drinks in synthetic resin containers and an odor control measure. Accordingly, the present invention can be used suitably not only in the field of packaging for various drinks, but also in the material processing industry handling material, such as synthetic resin, used for food packaging and containers.

Specific embodiments or examples implemented in the description of the embodiments only show technical features of the present invention and are not intended to limit the scope of the invention. Variations can be effected within the spirit of the present invention and the scope of the following claims.

What is claimed is:

1. A liquid composition, which is brought into contact with a given material, for use in quantifying an amount of components sorbed by the material,
wherein:
a hydrocarbon component, an ester component, an aldehyde component, an alcohol component, and a phenol component are contained as sorption components sorption amounts of which are quantified.

2. The liquid composition according to claim 1, wherein:
the hydrocarbon component contains at least one of terpene and an aromatic compound.

3. The liquid composition according to claim 1, wherein:
the hydrocarbon component contains α-pinene and/or limonene.

4. The liquid composition according to claim 1, wherein:
the ester component contains at least one of the following compounds: ethyl acetate, ethyl butyrate, ethyl caproate, ethyl caprate, ethyl laurate, ethyl myristate, and methyl anthranilate.

5. The liquid composition according to claim 1, wherein:
the aldehyde component contains at least one of the following compounds: citral, iso-butanal, n-butanal, n-octanal, n-dodecanal, and benzaldehyde.

6. The liquid composition according to claim 1, wherein:
the alcohol component contains at least one of the following compounds: linalol, geraniol, iso-butanol, n-butanol, n-octanol, n-dodecanol, and benzyl alcohol.

7. The liquid composition according to claim 1, wherein:
the phenol component contains phenol and/or cresol.

8. The liquid composition according to claim 1, wherein:
at least one compound having an evaporation energy-based solubility parameter of not less than 26.3 MPa^{1/2} is contained.

9. The liquid composition according to claim 8, wherein:
the compound having the solubility parameter of not less than 26.3 MPa^{1/2} is contained in the phenol component.

10. The liquid composition according to claim 8, wherein:
at least one compound having the evaporation energy-based solubility parameter of not less than 22.0 MPa^{1/2}. is further contained.

11. The liquid composition according to claim 10, wherein:
the compound having the solubility parameter of not less than 22.0 MPa^{1/2} is contained in at least any one of the ester component, the aldehyde component, and the alcohol component.

12. The liquid composition according to claim 8, wherein:
a compound contained in the alcohol component has the solubility parameter in a range from 20.1 MPa^{1/2} to 26.2 MPa^{1/2}.

13. The liquid composition according to claim 8, wherein:
a compound contained in the ester component has the solubility parameter in a range from 18.0 MPa^{1/2} to 23.6 MPa^{1/2}.

14. The liquid composition according to claim 8, wherein:
a compound contained in the aldehyde component has the solubility parameter in a range from 18.7 MPa^{1/2} to 23.8 MPa^{1/2}.

15. The liquid composition according to claim 8, wherein:
a compound contained in the hydrocarbon component has the solubility parameter in a range from 16.2 MPa^{1/2} to 18.5 MPa^{1/2}.

16. The liquid composition according to claim 1, wherein:
as a solvent dissolving the sorption components, at least any one of water, alcohol having not more than four carbon atoms, and saturated hydrocarbon having 5 to 17 carbon atoms is used.

17. The liquid composition according to claim 1, wherein:
as a solubilizing agent for the sorption components, a polyalcohol-type nonionic surfactant is further contained.

18. The liquid composition according to claim 17, wherein:
the polyalcohol-type nonionic surfactant is at least any one of sugar ester, glycerin fatty acid ester, sorbitan fatty acid ester, and propyleneglycol fatty acid ester.

19. The liquid composition according to claim 1, wherein:
compounds used as the sorption components are dissolved in a solvent so as to make a final concentration of each of the compounds in a range from 1 ppm to 100 ppm.

20. The liquid composition according to claim 1, wherein:
the material is synthetic resin.

21. The liquid composition according to claim 20, wherein:
the synthetic resin is any one of polyethylene terephthalate, polyethylene, ethylene-vinylalcohol copolymer, and polyamide.

22. A method for evaluating sorption behavior of flavor compounds, comprising:
a contact step of bringing the liquid composition according to claim 1 into contact with a given material; and
a sorption-amount measuring step of measuring an amount of a solute of the liquid composition sorbed by the material.

23. A method according to claim 22, wherein:
the material is in film form.
